(19) United States
(12) Patent Application Publication
(10) Pub. No.: US 2024/0398679 A1
(43) Pub. Date: Dec. 5, 2024

(54) SOLID COSMETIC FOR LIPS

(71) Applicant: Shiseido Company, Ltd., Tokyo (JP)

(72) Inventors: Keisuke HAYASHIDA, Tokyo (JP); Kiriko CHIBA, Tokyo (JP); Yusuke NAKANO, Tokyo (JP); Hiroshi OKAMOTO, Tokyo (JP)

(73) Assignee: Shiseido Company, Ltd., Tokyo (JP)

(21) Appl. No.: 18/270,132

(22) PCT Filed: Jan. 26, 2022
(86) PCT No.: PCT/JP2022/002839

§ 371 Date: Jun. 26, 2024

(30) Foreign Application Priority Data
Jan. 28, 2021 (JP) 2021-011974

(51) Int. Cl. A61K 8/37; A61K 8/02; A61K 8/31; A61K 8/92; A61Q 1/06

(52) U.S. Cl. CPC A61K 8/375; A61K 8/0229; A61K 8/31; A61K 8/92; A61Q 1/06

(57) ABSTRACT

The present invention is to provide a solid lip cosmetic which is excellent in secondary adhesion resistance effect and long lasting performance, and provides a good glossy finish.

A solid lip cosmetic which has a secondary adhesion resistance effect and provides a good glossy finish can be prepared by blending a copolymer containing a dimer acid ester, or a copolymer containing a dimer acid ester and a dimer acid ester as an oil component adhering to lips (adhesive oil component) and a hydrocarbon oil which is liquid at room temperature as an oil component seeping into the surface when the cosmetic is applied to the lips (seeping oil component).

### RELATED APPLICATION

The present application claims priority to Japanese Patent Application No. 2021-011974 filed Jan. 28, 2021, the disclosure of which is hereby incorporated by reference.

### FIELD OF THE INVENTION

The present invention relates to a solid lip cosmetic, in particular to an improvement of a solid lip cosmetic which is excellent in secondary adhesion resistance effect and long lasting performance, and provides a good glossy finish.

### BACKGROUND OF THE INVENTION

Conventional lip cosmetics have a problem of secondary adhesion, which is transference of the lipstick to things that come into contact with the lips such as a cup after application of the lipstick to the lips. In order to respond to this problem, lip cosmetics having so-called secondary adhesion resistance effect, which are less likely to cause secondary adhesion, have been developed.

For example, Patent Literatures 1 to 4 disclose a solid lip cosmetic using hydrogenated polyisobutene which adheres to lips and an organic silicone oil less compatible to hydrogenated polyisobutene at room temperature, which cosmetic is homogeneous in the form of a product but separates into hydrogenated polyisobutene and organic silicone oil when shared in use.

With lip cosmetics having the secondary adhesion resistance effect, hydrogenated polyisobutene adheres to the lips in use, and the organosilicon oil layer forms a film over the hydrogenated polyisobutene layer. Since this organosilicon oil is colorless and transparent, even if it is transferred onto a cup or other objects, the stain will not be noticeable, and the secondary adhesion resistance effect will be achieved.

However, when high viscosity oil agents such as hydrogenated polyisobutene and polyisobutylene are blended in large amounts as described above, there are drawbacks of difficulty in spreading during use and a sticky finish.

Then Patent Literature 5 discloses an oil-in-oil lip cosmetic with little stickiness and an excellent secondary adhesion resistance effect, in which a specific amount of phenyl-modified silicone is combined with high viscosity non-volatile ester oil which is not compatible with phenyl-modified silicone even at high temperature, dextrin fatty acid ester and volatile hydrocarbon.

However, the combination in Patent Literature 5 does not provide a solid lip cosmetic; and even when a solid lipstick is produced, the necessity of blending an incompatible oil component at high temperature may make the product inhomogeneous and brittle. The use of a phenyl-modified silicone may result in a too glossy finish, which remains to be a problem.

### CITATION LIST

### Patent Literature

[Patent Literature 1] Japanese Patent No. 4757950
[Patent Literature 2] Japanese Patent No. 5280490
[Patent Literature 3] Japanese Patent Laid-Open No. 2012-82188
[Patent Literature 4] Japanese Patent No. 6184454
[Patent Literature 5] Japanese Patent No. 6050679

### SUMMARY OF INVENTION

### Problem to be Solved by the Invention

The present invention has been made in view of the above problem in prior art and an object of the present invention is to provide a solid lip cosmetic which has an excellent secondary adhesion resistance effect and is excellent in long lasting performance, and provides a good glossy finish.

### Means to Solve the Problem

The present inventors have conducted intensive studies to solve the above problem, and as a result have found that a solid lip cosmetic which has a secondary adhesion resistance effect and provides a good glossy finish can be prepared by blending a copolymer containing a dimer acid ester, or a copolymer containing a dimer acid ester and a dimer acid ester as an oil component adhering to lips (adhesive oil component) and a hydrocarbon oil which is liquid at room temperature as an oil component seeping into the surface when the cosmetic is applied to the lips (seeping oil component).

To achieve the above object, the solid lip cosmetic of the present invention includes the following 6 aspects.

(1) The solid lip cosmetic comprises, as essential components, (A) 15 to 50% by mass of a copolymer containing a dimer acid ester (a1) or a copolymer containing a dimer acid ester (a1) and a dimer acid ester (a2), as an adhesive oil;
(B) 20 to 70% by mass of a non-volatile hydrocarbon oil which is liquid at room temperature; and
(C) 5 to 15% by mass of a wax.

It is preferable that the total amount of the component (A), the component (B) and the component (C) accounts for 80 to 100% by mass of the whole oil phase component.

It is preferable that the cosmetic further comprises (D) a color material.

Furthermore, the component (B) is compatible with the component (A) at 90° C. and separated from the component (A) at 25° C.

(2) In the above cosmetic, the component (B) has a viscosity of 100 mPa·s or less at room temperature.

(3) The above cosmetic comprises one or more of the hydrocarbon oils which are liquid at room temperature (B) selected from an olefin oligomer, squalane, liquid paraffin, light isoparaffin, heavy liquid paraffin and hydrogenated polydecene, and the ratio of (A):(B) is 1:0.5 to 1:4.

(4) In the above cosmetic, the copolymer containing a dimer acid ester of (A) is polyglyceryl-2 isostearate/dimer dilinoleate copolymer.

(5) In the above cosmetic, the dimer acid ester of (A) is dimer dilinoleyl dimer dilinoleate.

(6) In the above cosmetic, the amount of phenyl-modified silicone blended is 5% by mass or less.

### Effect of the Invention

The solid lip cosmetic of the present invention is prepared by blending a specific amount of (A) (a1) a copolymer containing a dimer acid ester or (a1) a copolymer containing a dimer acid ester and (a2) a dimer acid ester, (B) a hydrocarbon oil which is liquid at room temperature and (C) a wax. Thus, the solid lip cosmetic of the present invention has a secondary adhesion resistance effect, achieves a good glossy finish and is good in long lasting performance.

### DESCRIPTION OF THE PREFERRED EMBODIMENTS

The solid lip cosmetic of the present invention is made of (A) (a1) a copolymer containing a dimer acid ester or (a1) a copolymer containing a dimer acid ester and (a2) a dimer acid ester, (B) a hydrocarbon oil which is liquid at room temperature and (C) a wax.

Hereinafter the respective components will be described in detail.

### (A) (a1) Copolymer Containing Dimer Acid Ester/(a2) Dimer Acid Ester

The copolymer containing a dimer acid ester (a1) is a high viscosity non-volatile oil. To improve the secondary adhesion resistance effect, those compatible with phenyl-modified silicone (B) at 90° C. and separates therefrom at 25° C. are selected. Specific examples thereof include a dimer dilinoleate copolymer. The dimer acid ester (a2) is liquid fatty acid ester containing, as the main component, a dibasic acid of C36 dicarboxylic acid formed by dimerization of C18 unsaturated fatty acid made of vegetable oils and fats as the raw material, and a monobasic acid and a tribasic acid.

Examples of copolymers containing a dimer acid ester (a1) include a commercially available product, HAILUCENT ISDA (made by Kokyu Alcohol Kogyo Co., Ltd.). Examples of dimer acid esters (a2) include a commercially available product, LUSPLAN DD-DA (made by Nippon Fine Chemical Co., Ltd.).

It is preferable that the amount of the component (A) blended is 15 to 50% by mass based on the whole cosmetic.

The amount of the component (A) blended is preferably 20 to 40% by mass, and more preferably 25 to 35% by mass from the viewpoint of adhesiveness to lips and durability of the cosmetic effect.

### (B) Hydrocarbon Oil Liquid at Room Temperature

It is essential that the hydrocarbon oil which is liquid at room temperature (B) has a viscosity of 100 mPa·s or less and is non-volatile. The hydrocarbon oil may be linear or branched and may be used without limitation as long as it is usually used in cosmetics. In the present invention, viscosity is measured by a B-type viscometer TVB-10 (made by Yamato Scientific Co., Ltd.) at 25° C. with spindle No. M1 at 1,000 rpm.

Furthermore, when the hydrocarbon oil is mixed with the copolymer containing a dimer acid ester (a1), or the copolymer containing a dimer acid ester (a1) and the dimer acid ester (a2), the hydrocarbon oil is separated therefrom at 25° C.

### Condition of Separation

In the present invention, whether the components are “separated” or not is observed under the following conditions.

### (Measurement Conditions)

The component (A) and the component (B) are heated to 90° C. and mixed with stirring at (A):(B)=1:1 (mass ratio) and allowed to stand. Those which are separated into two homogeneous layers at the boundary when the temperature of the mixture reaches 25° C. are rated as “separated” and those which are transparent and have no boundary are rated as “not separated.” Those which are opaque without visible boundaries are rated as cloudy.

In the present invention, whether components are “compatible” or not is determined based on the following condition.

Components are determined as compatible when the mixture is transparent without visible boundaries at 90° C., in other words, “not separated.”

Examples of hydrocarbon oils liquid at room temperature (B) include an olefin oligomer, squalane, liquid paraffin, light isoparaffin, heavy liquid isoparaffin and hydrogenated polydecene.

Examples of hydrocarbon oils liquid at room temperature (B) also include a commercially available product NOMUCOAT HP100 (made by The Nisshin OilliO Group Ltd.) and squalane (made by Kuraray Co., Ltd.).

The amount of the component (B) blended is 20 to 70% by mass based on the whole cosmetic. The amount of the component (B) blended is preferably 30 to 55% by mass, and more preferably 35 to 50% by mass.

When the amount of the component (B) blended is less than 20% by mass, the cosmetic is less likely to be separated when applied and there is little secondary adhesion resistance effect. Furthermore, when the amount of the component (B) blended is more than 70% by mass, the amount of other components blended is reduced, and there is little secondary adhesion resistance effect.

It is preferable that the component (B) has a viscosity of 100 mPa·s or less. When the component (B) has a viscosity of more than 100 mPa·s, the cosmetic is less likely to be separated when applied and does not last long. The component (B) has a viscosity of more preferably 50 mPa·s or less. It is also preferable that the component (B) is a non-volatile oil component. Volatile oil components do not function as a seeping oil and reduces the secondary adhesion resistance effect and long lasting performance.

The ratio of blending between the component (A) and the component (B) is 1:0.5 to 1:4. The ratio of blending is preferably 1:0.8 to 1:3.5 and more preferably 1:1 to 1:2. When the ratio of blending is in that range, the component (A) and the component (B) are compatible at 90° C. and separate at 25° C.

### (C) Wax

The wax (C) blended to the solid lip cosmetic of the present invention is not particularly limited as long as it is usually blended in cosmetics.

It is preferable that the wax (C) used in the present invention is compatible with the component (A) and the component (B) at high temperature and separate therefrom at room temperature.

Examples of wax (C) used in the present invention include carnauba wax, candelilla wax, polyethylene wax, beeswax, ceresin, microcrystalline wax, solid paraffin, Japan wax, beeswax and polyethylene wax.

The amount of the component (C) blended is preferably 5 to 15% by mass based on the total amount of the cosmetic.

The amount of the component (C) blended is more preferably 7 to 12% by mass. When the amount of the component (C) blended is 5% by mass or less, the resultant may be difficult to be solidified. When the amount of the component (C) blended is more than 15%, the resulting cosmetic may be difficult to be spread and less glossy.

The solid lip cosmetic of the present invention may include a component usually used for solid lip cosmetics as an optional component in addition to the above essential components (A) to (C).

It is also preferable to blend a color material (D) in the solid lip cosmetic of the present invention. A color material usually used for solid lip cosmetics may be blended as the color material.

The color material (D) may be one usually used for cosmetics, and may be in the form of powder or lake (with oil kneaded into it). The color material (D) may be an inorganic pigment, an organic pigment or a pearl pigment.

In this case it is desired that the color material is dissolved or dispersed in the component (A) which adheres to lips and is not dissolved or dispersed in the component (B) which is the seeping oil component. This is because when the color material is also dispersed in the component (B), the secondary adhesion resistance effect is reduced.

A pigment, a pearl pigment, a lame agent and an agent prepared by chelating them, which are usually blended in cosmetics, may be used as the color material (D).

Examples of color materials include an inorganic white pigment (titanium dioxide, zinc oxide), an inorganic red pigment (iron oxide (red iron oxide), iron titanate), an inorganic brown pigment (γ-iron oxide), an inorganic yellow pigment (yellow iron oxide, yellow ocher), an inorganic black pigment (black iron oxide, carbon, titanium suboxide), an inorganic purple pigment (mango violet, cobalt violet), an inorganic green pigment (chromium oxide, chromium hydroxide, cobalt titanate), an inorganic blue pigment (ultramarine, Prussian blue), a pearl pigment (titanium oxide-coated mica, titanium oxide-coated bismuth oxychloride, titanium oxide-coated talc, colored titanium oxide-coated mica, bismuth oxychloride, fish scale guanine), a metal powder pigment (aluminum powder, copper powder), an organic pigment (Red No. 202, Red No. 205, Red No. 220, Red No. 228, Red No. 405, Orange No. 203, Orange No. 204, Yellow No. 205, Yellow No. 40, Blue No. 404), zirconium, barium, aluminum lake organic pigment (Red No. 3, Red No. 104, Red No. 227, Red No. 401, Orange No. 205, Yellow No. 4, Yellow No. 20, Green No. 3, Blue No. 1), a natural coloring (chlorophyll, carcinoid (β-carotene), carthamin, cochineal, calcone, curcumin, betanine, flavonol, flavone, anthocyanidin, anthraquinone, naphthoquinone) and a functional pigment (boron nitride, photochromic pigment, synthetic fluorphlogopite, iron-containing synthetic fluorphlogopite and hybrid fine powder).

The amount of the component (D) blended is preferably 0.1 to 15% by mass based on the whole cosmetic. The amount of the component (D) blended is more preferably 4 to 10% by mass. When the amount of the component (D) blended is less than 0.1% by mass, the secondary adhesion resistance effect is unlikely to be felt in some cases. When the amount of the component (D) blended is more than 15% by mass, the amount of the components (A) to (C) is relatively reduced, and thus the secondary adhesion resistance effect may be reduced.

The solid lip cosmetic of the present invention may be prepared without so-called “binder oil.” The binder oil allows the adhesive oil component and the steeping oil component to be compatibilized at high temperature. In the present invention, the cosmetic may include, but does not necessarily include, the binder oil.

Examples of binder oils include diisostearyl malate, neopentyl glycol dicaprate, triethylhexanoin, pentaerythrityl tetraethylhexanonate, caprylic/capric triglyceride. It is preferable to blend 0 to 20% by mass of the binder oil.

In addition to the above components, a component used for usual lip cosmetics such as an oil agent, powder, a polymer compound, a moisturizer, a perfume, an antioxidant, a preservative and a beauty ingredient may be blended in the solid lip cosmetic of the present invention to the extent that the effect of the present invention is not lost.

In the present invention, it is preferable that when phenyl-modified silicone is blended to the cosmetic, the amount is less than 5% by mass based on the whole cosmetic. This is because when the amount of phenyl-modified silicone blended is more than 5% by mass, compatibility of the oil components is reduced and the cosmetic is less stable.

In the present invention it is preferable that the amount of powder added is 30% by mass or less. The amount of powder added is more preferably 20% by mass or less. When the amount of powder added is more than 30% by mass, the amount of the components (A) to (C) blended is relatively reduced, and thus the secondary adhesion resistance effect may be reduced.

Examples of powders include a spherical powder, a flaky powder.

Examples of spherical powders include a spherical resin powder such as methyl polymethacrylate, organopolysiloxane elastomer, polystyrene, polyamide resin (nylon), polyethylene, a copolymer of styrene and acrylic acid, benzoguanamine resin, polytetrafluoroethylene and a silicone resin.

Examples of flaky powders include inorganic powder such as mica, synthetic mica, talc, sericite, aluminum oxide, magnesium oxide, zirconium oxide, magnesium carbonate, calcium carbonate, calcium sulfate, chromium oxide, chromium hydroxide, aluminum silicate, magnesium silicate, aluminum magnesium silicate, kaolin, silicon carbide, barium sulfate, bentonite, smectite and boron nitride, an organic powder such as N-acyl lysine, and a composite powder such as fine particle titanium oxide-coated titanated mica, fine particle zinc oxide-coated titanated mica, and barium sulfate-coated titanated mica.

A hydrophilized powder may also be used as the powder such as a pigment, a pearl pigment and a lame agent. Powder prepared by a hydrophilization treatment known in the art may be used as the hydrophilized powder. Both organic treatment and inorganic treatment may be used as the hydrophilization treatment. The hydrophilizing agent is not particularly limited and examples thereof include polyhydric alcohol, polysaccharide, water-soluble polymer, metal alkoxide and water glass.

It is preferable that the components of the solid lip cosmetic of the present invention are designed so that the components are in a homogeneous phase without separation throughout the process of production.

The solid lip cosmetic of the present invention may be applied to a lipstick, a lip gloss, a lip primer, an overcoat for lipsticks, and a lip cream. In particular, a lipstick in the form of a solid stick is preferred.

### EXAMPLES

The present invention will be described with reference to Examples below, but the present invention is not limited by those Examples. The amount blended is in % by mass unless otherwise specified.

First, the present inventors observed compatibility of a copolymer containing a dimer acid ester (a1) (HAILUCENT ISDA) with various oils. The results are shown in the following Table 1.

### (Conditions of Measurement of Mixed State)

The component (A) and the component (B) were heated to 90° C. and mixed with stirring at (A):(B)=1:1 (mass ratio), and then the condition was observed. Next the mixture was allowed to stand, and the condition was also observed when the temperature of the mixture reached 25° C. Those which were separated into two homogeneous layers at the boundary were rated as “separated.” Those which were transparent and had no boundary were rated as “compatible.” Those which were opaque without visible boundaries were rated as “cloudy”.

TABLE 1

| Type of oil | Oil component | 90° C. | 25° C. |
|---|---|---|---|
| Ester oil | Di(phytosteryl × 2-octyldodecyl) | Compatible | Separated |
| | N-lauroyl-L-glutamate*1 | | |
| Ester oil | (Phytosteryl/isostearyl/ | Cloudy | Separated |
| | cetyl/stearyl/ | | |
| | behenyl) dimer dilinoleate*2 | | |
| Ester oil | Caprylic/capric triglyceride*3 | Compatible | Cloudy |
| Ester oil | Dipentaerythrityl | Compatible | Cloudy |
| | hexahydroxystearate*4 | | |
| Higher alcohol | 2-Octyldodecanol | Compatible | Cloudy |
| Hydrocarbon oil | Olefin oligomer 30 | Compatible | Separated |
| Hydrocarbon oil | Squalane | Compatible | Separated |

*1ELDEW PS-203 (Ajinomoto Co., Inc.)
*2Plandool PB (Nippon Fine Chemical Co., Ltd.)
*3ODO (The Nisshin OilliO Group Ltd.)
*4COSMOL 168M (The Nisshin OilliO Group Ltd.)

Table 1 shows that a hydrocarbon oil which is liquid at room temperature is an example of oil components which are compatible with a copolymer containing a dimer acid ester (HAILUCENT ISDA) (a1) at 90° C. and separated therefrom at 25° C. when mixed therewith.

Next, compatibility of various hydrocarbon oils with different types of copolymers containing a dimer acid ester (a1) and/or dimer acid esters (a2) was assessed. The results are shown in Table 2.

TABLE 2

| Compatibility of oil component at 90° C. and 25° C. | | Hydrocarbon oil | | |
|---|---|---|---|---|
| | | | | Hydrogenated |
| | | Olefin | | polyisobutene(28 |
| (90° C./25° C.) | | oligomer | Squalane | mPa · s) |
| High | Polyglyceryl-2 isostearate/ | Compatible/ | Compatible/ | Compatible/ |
| viscosity | dimer dilinoleate | separated | separated | separated |
| ester oil | copolymer*5 | | | |
| | Dimer dilinoleyl dimer | Compatible/ | Compatible/ | Compatible/ |
| | dilinoleate * 6 | compatible | compatible | compatible |
| | 1:1 Mixture of polyglyceryl-2 | Compatible/ | Compatible/ | Compatible/ |
| | isostearate/dimer | separated | separated | separated |
| | dilinoleate copolymer and | | | |
| | dimer dilinoleyl dimer | | | |
| | dilinoleate | | | |
| | Di(octyldodecyl/phytosteryl/ | Compatible/ | Compatible/ | Compatible/ |
| | behenyl) lauroyl | solidified | solidified | solidified |
| | glutamate * 7 | | | |
| | 1:1 Mixture of polyglyceryl-2 | Compatible/ | Compatible/ | Compatible/ |
| | isostearate/dimer | solidified | solidified | solidified |
| | dilinoleate copolymer and | | | |
| | di(octyldodecyl/phytosteryl/ | | | |
| | behenyl) lauroyl | | | |
| | glutamate | | | |

*5HAILUCENT ISDA (Kokyu Alcohol Kogyo Co., Ltd.)
* 6 LUSPLAN DD-DA7 (Nippon Fine Chemical Co., Ltd.)
* 7 ELDEW PS-306 (Ajinomoto Co., Inc.)

The results of Table 2 show that only the copolymer containing a dimer acid ester (a1) and the 1:1 mixture of the copolymer containing a dimer acid ester (a1) and the dimer acid ester (a2) were compatible with the hydrocarbon at 90° C. and separated therefrom at 25° C. Thus, a combination of the copolymer containing a dimer acid ester (a1) with a hydrocarbon oil which is liquid at room temperature (B) and a combination of a mixture of the copolymer containing a dimer acid ester (a1) and the dimer acid ester (a2) with a hydrocarbon oil which is liquid at room temperature (B) are preferred.

The present inventors prepared samples (solid lipsticks) having the composition shown in the tables by a usual method. The respective samples were evaluated according to the following criteria.

### (Method of Evaluating Moldability)

Upon removal of the lipstick from the mold for molding, evaluation was made according to the following criteria.
A: Lipstick not chipped, or not adhering to mold
C: Lipstick chipped, or adhering to mold

### (Method of Evaluating Secondary Adhesion Resistance)

A practical usability test was performed by a panel of 10 experts. The secondary adhesion resistance effect when the sample was applied to lips was assessed (scored) on a scale of 1 to 5 based on the following grading scale. From the average score, determination was made based on the following evaluation criteria.

### (Score)

5: Very good
4: Good
3: Moderate
2: Poor
1: Bad

### (Evaluation Criteria)

S: Average score of 4.0 or more
A: Average score of 3.5 or more and less than 4.0
B: Average score of 2.5 or more and less than 3.5
C: Average score of less than 2.5

### (Method of Evaluating Gloss)

A practical usability test was performed by a panel of 10 experts. Gloss when the sample was applied to lips was assessed (scored) on a scale of 1 to 3 based on the following grading scale. From the average score, determination was made based on the following evaluation criteria.

### (Score)

3: Less glossy than reference formulation
2: Slightly less glossy than reference formulation
1: As glossy as reference formulation

### (Evaluation Criteria)

A: Average score of 2.0 or more
B: Average score of 1.5 or more and less than 2.0
C: Average score of less than 1.5

[Reference formulation]

| Component | Amount |
|---|---|
| (A)Polyglyceryl-2 isostearate/dimer dilinoleate | 30 |
| copolymer*5 | |
| (B)Phenyl-modified silicone | 50 |
| (C)Wax | 10 |
| (D)Color material | 10 |

### (Method of Evaluating Long Lasting Performance of Cosmetic)

A practical usability test was performed by a panel of 10 experts. Long lasting performance of the cosmetic two hours after the sample was applied to lips was assessed (scored) on a scale of 1 to 5 based on the following grading scale. From the average score, determination was made based on the following evaluation criteria.

### (Score)

5: Very good
4: Good
3: Moderate
2: Poor
1: Bad

### (Evaluation Criteria)

S: Average score of 4.0 or more
A: Average score of 3.5 or more and less than 4.0
B: Average score of 2.5 or more and less than 3.5
C: Average score of less than 2.5

The present inventors investigated the viscosity of the component (B) that can be mixed.

The results are shown in Table 3.

TABLE 3

| | — | 27.7 mPa · s |
|---|---|---|
| Viscosity of hydrocarbon oil | Volatile | Non-volatile |
| (A) Polyglyceryl-2 isostearate/dimer | 30 | 30 |
| dilinoleate copolymer*5 | | |
| (B) Hydrocarbon oil | 50 | 50 |
| (C) Wax | 10 | 10 |
| (D) Color material | 10 | 10 |
| Moldability | A | A |
| Secondary adhesion resistance effect | C | A |
| Gloss | C | A |
| Long lasting performance | C | A |

The results of Table 3 above show that it is necessary to use a non-volatile hydrocarbon as the component (B). Preferable usability cannot be achieved with volatile hydrocarbon oil. The results also suggest that when the non-volatile hydrocarbon oil has a viscosity of 100 mPa·s or less, the cosmetic is excellent in secondary adhesion resistance effect and good gloss and excellent long lasting performance can be obtained. The non-volatile hydrocarbon oil has a viscosity of preferably 50 mPa·s or less.

The present inventors investigated the ratio of mixing between the component (A) and the component (B).

The results are shown in Table 4.

TABLE 4

| Test Example | 1-1 | 1-2 | 1-3 | 1-4 | 1-5 | 1-6 |
|---|---|---|---|---|---|---|
| (A) Polyglyceryl-2 isostearate/dimer | 44.6 | 40 | 40 | 34.1 | 30 | 29.4 |
| dilinoleate copolymer*5 | | | | | | |
| (B)Olefin oligomer | 35.7 | 45.3 | | 51.2 | | 55.9 |
| (B)Squalane | | | 45.3 | | 55.3 | |
| (B)Hydrogenated | | | | | | 53.5 |
| polyisobutene(28 mPa · s) | | | | | | |
| (C) Wax | 15 | 10 | 10 | 10 | 10 | 10 |
| (D) Color material | 4.7 | 4.7 | 4.7 | 4.7 | 4.7 | 4.7 |
| (A):(B) | 1:0.8 | 1:1.1 | 1:1.1 | 1:1.5 | 1:1.8 | 1:1.9 |
| Moldability | A | A | A | A | A | A |
| Secondary adhesion resistance effect | A | A | A | A | A | A |
| Gloss | A | A | A | A | A | A |
| Long lasting performance | A | A | A | A | A | A |
| Test Example | 1-8 | 1-9 | 1-10 | 1-11 | 1-12 | 1-13 |
| (A) Polyglyceryl-2 isostearate/dimer | 25.1 | 21 | 19 | 60.9 | 13 | 16.3 |
| dilinoleate copolymer*5 | | | | | | |
| (B)Olefin oligomer | | | | | 72.3 | |
| (B)Squalane | 60.2 | | | 24.4 | | |
| (B)Hydrogenated | | 64.3 | 61.3 | | | 65.3 |
| polyisobutene(28 mPa · s) | | | | | | |
| (C) Wax | 10 | 10 | 15 | 10 | 10 | 13.7 |
| (D) Color material | 4.7 | 4.7 | 4.7 | 4.7 | 4.7 | 4.7 |
| (A):(B) | 1:2.3 | 1:3.1 | 1:3.2 | 1:0.4 | 1:5.6 | 1:4.0 |
| Moldability | A | A | A | C | A | A |
| Secondary adhesion resistance effect | A | A | A | C | C | A |
| Gloss | A | A | A | C | A | A |
| Long lasting performance | A | A | A | C | C | A |

The results of Test Examples 1-1 to 1-10 in Table 4 show that the amount of the copolymer containing a dimer acid ester (A) blended is preferably 15 to 50% by mass. Furthermore, it is preferable that the amount of blended hydrocarbon which is liquid at room temperature (B) is 20 to 70% by mass. The same Test Examples suggest that when a blending ratio between the component (A) and the component (B) is 1:0.5 to 1:4, a solid lip cosmetic with a secondary adhesion resistance effect and good usability can be obtained.

The present inventors studied to find an adhesive oil component other than the copolymer containing a dimer acid ester (a1). The results are shown in Table 5.

TABLE 5

| | Test Example | 2-1 | 2-2 | 2-3 | 2-4 | 2-5 | 2-6 | 2-7 | 2-8 | 2-9 | 2-10 |
|---|---|---|---|---|---|---|---|---|---|---|---|
| (A) | Polyglyceryl-2 isostearate/dimer | 8 | 15 | 20 | 30 | 15 | 30 | | | | |
| | dilinoleate copolymer*5 | | | | | | | | | | |
| | Dimer dilinoleyl dimer dilinoleate | 8 | 15 | 20 | 10 | | | 15 | 30 | | |
| | * 6 | | | | | | | | | | |
| | Di(octyldodecyl/phytosteryl/ | | | | | | | | | 15 | 30 |
| | behenyl) lauroyl glutamate * 7 | | | | | | | | | | |
| (B) | Olefin oligomer 30 | | 53 | 43 | 43 | | 53 | 68 | 53 | 68 | 53 |
| | Parleam 6 | 67 | | | | 68 | | | | | |
| (C) | Wax | 12 | 12 | 12 | 12 | 12 | 12 | 12 | 12 | 12 | 12 |
| (D) | Color material | 5 | 5 | 5 | 5 | 5 | 5 | 5 | 5 | 5 | 5 |

| | | | | | | | | | | |
|---|---|---|---|---|---|---|---|---|---|---|
| Moldability | A | A | A | A | A | A | A | A | A | A |
| Secondary adhesion resistance effect | B | A | A | A | B | A | C | C | C | C |
| Gloss | A | A | A | A | A | A | A | A | C | C |
| Long lasting performance | B | A | A | A | B | A | C | C | C | C |

A comparison between Test Examples 2-1 to 2-6 and 2-7 to 2-10 in Table 5 show that it is essential to blend the copolymer containing a dimer acid ester (a1), i.e., polyglyceryl-2 isostearate/dimer dilinoleate copolymer, as an adhesive oil component.

Furthermore, a comparison between Test Examples 2-1 to 2-4 and 2-7, 2-8 show that the dimer acid ester (a2) also works as an adhesive oil component when combined with the copolymer containing a dimer acid ester (a1). In that case, it is necessary that the copolymer containing a dimer acid ester (a1) accounts for 50% by mass or more of the adhesive oil.

### Formulation Example 1: Lip Stick

Component Compounding Amount (% by mass)

Polygryceryl-2 isostearated/dimer dilinoleate copolymer*5 30

Squalane 44.9

Polyethylene wax 10

Color material 10

Pearl pigment 5

Niacinamide 0.1

### Formulation Example 2: Lip Stick

Component Compounding Amount (% by mass)

Polygryceryl-2 isostearated/dimer dilinoleate copolymer*5 15

Dimer dilinoleyl dimer dilinoleate*6 15

Hydrogenated polyisobutene 45

Polyethylene wax 10

Color material 10

Pearl pigment 5

### Formulation Example 3: Lip Stick

Component Compounding Amount (% by mass)

Polygryceryl-2 isostearated/dimer dilinoleate copolymer*5 30

Squalane Remaining amount

Polyethylene wax 10

Color material 10

Pearl pigment 5

Niacinamide 5

What is claimed is:

1. A solid lip cosmetic comprising:
(A) 15 to 50% by mass of a copolymer containing a dimer acid ester (a1) or a copolymer containing a dimer acid ester (a1) and a dimer acid ester (a2), as an adhesive oil;
(B) 20 to 70% by mass of a non-volatile hydrocarbon oil which is liquid at room temperature, as a seeping oil; and
(C) 5 to 15% by mass of a wax,
wherein the copolymer containing a dimer acid ester (a1) accounts for 50% by mass or more of the component (A), and
a total amount of the components (A) to (C) accounts for 80 to 100% by mass of the whole oil phase component.

2. The solid lip cosmetic according to claim 1, wherein the component (B) has a viscosity of 100 mPa·s or less at room temperature.

3. The solid lip cosmetic according to claim 1 or 2, wherein the solid lip cosmetic comprises one or more of the hydrocarbon oils which are liquid at room temperature (B) selected from an olefin oligomer, squalane, liquid paraffin, light isoparaffin, heavy liquid paraffin and hydrogenated polydecene, and
a ratio of (A):(B) is 1:0.5 to 1:4.

4. The solid lip cosmetic according to any one of claims 1 to 3, wherein the copolymer containing a dimer acid ester (a1) is polyglyceryl-2 isostearate/dimer dilinoleate copolymer.

5. The solid lip cosmetic according to any one of claims 1 to 4, wherein the dimer acid ester (a2) is dimer dilinoleyl dimer dilinoleate.

6. The solid lip cosmetic according to any one of claims 1 to 5, wherein an amount of phenyl-modified silicone blended is 5% by mass or less.
